SEQUENCE LISTING (29 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 5 aa, Homo sapiens
SYTMH

SEQ ID NO: 4 - protein, 17 aa, Homo sapiens
FISYDGNNKYYADSVKG

SEQ ID NO: 5 - protein, 9 aa, Homo sapiens
TGWLGPFDY

SEQ ID NO: 6 - protein, 12 aa, Homo sapiens
RASQSVGSSYLA

SEQ ID NO: 7 - protein, 7 aa, Homo sapiens
GAFSRAT

SEQ ID NO: 8 - protein, 9 aa, Homo sapiens
QQYGSSPWT

SEQ ID NO: 9 - protein, 118 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 10 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 11 - protein, 448 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 447 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 215 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 10 aa, Homo sapiens
GFTFSSYGMH

SEQ ID NO: 15 - protein, 15 aa, Homo sapiens
VIWYDGSNKYYADSV

SEQ ID NO: 16 - protein, 16 aa, Homo sapiens
DPRGATLYYYYYGMDV

SEQ ID NO: 17 - protein, 11 aa, Homo sapiens
RASQSINSYLD

SEQ ID NO: 18 - protein, 7 aa, Homo sapiens
AASSLQS

SEQ ID NO: 19 - protein, 9 aa, Homo sapiens
QQYYSTPFT

SEQ ID NO: 20 - protein, 176 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 21 - protein, 146 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 22 - protein, 451 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 450 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 24 - protein, 214 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 9 aa, Simian immunodeficiency virus
RPKVPLRTM

SEQ ID NO: 26 - protein, 9 aa, Simian immunodeficiency virus
LNMADKKET

SEQ ID NO: 27 - protein, 9 aa, Simian immunodeficiency virus
GPRKPIKCW

SEQ ID NO: 28 - protein, 330 aa, Homo sapiens [sequence omitted]

SEQ ID NO: 29 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2022/0127363 A1
(43) Pub. Date: Apr. 28, 2022

(54) USE OF ANTI-CTLA-4 ANTIBODIES WITH ENHANCED ADCC TO ENHANCE IMMUNE RESPONSE TO A VACCINE

(71) Applicant: BRISTOL-MYERS SQUIBB COMPANY, Princeton, NJ (US)

(72) Inventors: John T. LOFFREDO, Yardley, PA (US); Katherine E. LEWIS, Lake Forest Park, WA (US); Robert F. GRAZIANO, Frenchtown, NJ (US); Alan J. KORMAN, Piedmont, CA (US)

(21) Appl. No.: 17/575,498

(22) Filed: Jan. 13, 2022

Related U.S. Application Data
(63) Continuation of application No. 16/488,118, filed on Aug. 22, 2019.
(60) Provisional application No. 62/468,527, filed on Mar. 8, 2017.
(60) Provisional application No. 62/464,738, filed on Feb. 28, 2017.

(51) Int. Cl. C07K 16/28; A61K 39/21; A61K 39/395

(52) U.S. Cl. CPC C07K 16/2818; A61K 2039/505; A61K 39/3955; A61K 39/21

(57) ABSTRACT

The present invention provides methods of enhancing immune response to a vaccine using variant forms of anti-CTLA-4 antibodies having enhanced ADCC activity. Variant anti-CTLA-4 antibodies for use in the present invention include nonfucosylated ipilimumab.

### FIELD OF THE INVENTION

The present application discloses methods of enhancing immune response to a vaccine, and specifically use of an immunomodulatory antibody as a vaccine adjuvant.

### BACKGROUND OF THE INVENTION

Vaccines are intended to elicit an immune response to an agent, such as a pathogen or tumor cells. However, vaccines don't always elicit an immune response. Adjuvants are compounds that are administered in conjunction with vaccines to enhance immune response, but typically enhance humoral rather than cellular immunity, which is particularly critical to the effectiveness of cancer vaccines. Ikeda et al. (2004) Cancer Sci. 95:697. Antibodies to immunomodulatory receptors have been proposed as vaccine adjuvants. See Keler et al. (2003) J. Immunol. 171:6251; Ponte et al. (2010) Immunol. 130:231; Kwek et al. (2012) Nat. Rev. Cancer 12:289; WO 2009/100140; WO 2014/089113. See also Clinical Trial NCT00113984 (using anti-CTLA-4 antibody ipilimumab as a potential adjuvant for a therapeutic vaccine for prostate cancer.) However, existing adjuvants are not always completely effective.

The need exists for agents with improved vaccine adjuvant activity. Such improved adjuvants would ideally enhance the magnitude of immune response to a vaccine at a given dose of the vaccine, reduce the amount of vaccine needed to achieve a desired level of immune response, and/or increase the duration of an immune response. Such agents would preferably enhance not only humoral immune response, but also cellular immune response.

### SUMMARY OF THE INVENTION

The present invention provides methods of enhancing the immune response to a vaccine using an anti-CTLA-4 antibody with enhanced ADCC activity. The anti-CTLA-4 antibody with enhanced ADCC activity of the invention is administered in conjunction with a vaccine, such as a tumor vaccine.

In one embodiment, the anti-CTLA-4 antibody with enhanced ADCC activity comprises the CDRH1, CDRH2, CDRH3, CDRL1, CDRL2 and CDRL3 sequences of SEQ ID NOs: 3-8, respectively. In another embodiment, the anti-CTLA-4 antibody with enhanced ADCC activity comprises the V_{H} and V_{L} sequences of SEQ ID NOs: 9 and 10, respectively. In a further embodiment, the anti-CTLA-4 antibody with enhanced ADCC activity comprises the HC sequence of SEQ ID NO: 11 or 12, and the LC sequence of SEQ ID NO: 13.

In an alternative embodiment, the anti-CTLA-4 antibody with enhanced ADCC activity comprises the CDRH1, CDRH2, CDRH3, CDRL1, CDRL2 and CDRL3 sequences of SEQ ID NOs: 14-19, respectively. In another embodiment, the anti-CTLA-4 antibody with enhanced ADCC activity comprises the V_{H} and V_{L} sequences of SEQ ID NOs: 20 and 21, respectively. In a further embodiment, the anti-CTLA-4 antibody with enhanced ADCC activity comprises the HC sequence of SEQ ID NO: 22 or 23, and the LC sequence of SEQ ID NO: 24.

Enhanced ADCC is measured with reference to the ADCC activity of ipilimumab. In various embodiments the anti-CTLA-4 antibody of the present invention exhibits 2-fold, 10-fold or greater ADCC compared with ipilimumab. In one embodiment, ADCC is measured by the NK92 cell mediated lysis assay described at Example 2. In one embodiment, the anti-CTLA-4 antibody with enhanced ADCC of the present invention exhibits an EC50 that is at least two-fold lower than the EC50 for ipilimumab in the assay described at Example 2. In another embodiment, the anti-CTLA-4 antibody with enhanced ADCC of the present invention exhibits an EC50 that is at least ten-fold lower than the EC50 for ipilimumab in the assay described at Example 2.

In other embodiments, the anti-CTLA-4 antibody with enhanced ADCC of the present invention has reduced fucosylation, or is hypofucosylated or nonfucosylated. In further embodiments, the anti-CTLA-4 antibody with enhanced ADCC of the present invention comprises i) one or more amino acid mutations to the Fc region to enhance FcγR binding and optionally ii) reduced or eliminated fucosylation.

In one embodiment, the anti-CTLA-4 antibody with enhanced ADCC of the present invention is ipilimumab with reduced fucosylation. In another embodiment, the anti-CTLA-4 antibody with enhanced ADCC of the present invention is hypofucosylated ipilimumab. In yet another embodiment, the anti-CTLA-4 antibody with enhanced ADCC of the present invention is nonfucosylated ipilimumab.

In another embodiment, the anti-CTLA-4 antibody with enhanced ADCC of the present invention is tremelimumab with reduced fucosylation. In another embodiment, the anti-CTLA-4 antibody with enhanced ADCC of the present invention is hypofucosylated tremelimumab. In yet another embodiment, the anti-CTLA-4 antibody with enhanced ADCC of the present invention is nonfucosylated tremelimumab.

In some embodiments, the anti-CTLA-4 antibody with enhanced ADCC of the present invention includes at least one amino acid mutation that enhances binding to activating Fcγ receptors (FcγR), such as a mutation, or cluster of mutations, selected from the group consisting of i) G236A, ii) S239D, iii) F243L, iv) E333A, v) G236A/I332E, vi) S239D/I332E, vii) S267E/H268F, viii) S267E/S324T, ix) H268F/S324T, x) G236A/S239D/I332E, xi) S239D/A330L/I332E, xii) S267E/H268F/S324T and xiii) G236A/S239D/A330L/I332E. In a further embodiment, the anti-human CTLA-4 antibody with enhanced ADCC activity comprising one or more amino acids that enhance ADCC also has reduced fucosylation or is hypofucosylated or nonfucosylated.

### BRIEF DESCRIPTION OF THE DRAWINGS

The experimental results provided in the drawings are derived from three independent replicates (Replicate A, Replicate B and Replicate C) of the experiments in Mauritian cynomolgus macaques described at Example 1. Replicate A, which involved four cynos/group, provided the samples used to obtain the data displayed at FIGS. 1A, 2A, 3A, 4A, 5A, 7A, 8A, and 9A. Replicate B, which involved six cynos/group, provided the samples used to obtain the data displayed at FIGS. 1B, 2B, 3B, 4B, 5B, 6A (there being no Nef LT9 data from Replicate A), 7B, 8B, 9B and 11. Replicate C, which involved six cynos/group, provided the samples used to obtain the data displayed at FIGS. 1C, 2C, 3C, 4C, 5C, 6B (there being no Nef LT9 data from Replicate A), 7C, 8C, and 9C. Although specific numerical values for data points may vary between replicates due to minor differences in the separate experimental protocols (e.g. comparing replicates A and B to replicate C), the qualitative trends, and thus the relevant scientific conclusions, are the same.

For the nonfucosylated anti-CTLA-4 antibodies, replicates B and C employed anti-human CTLA-4 mAb ipilimumab (YERVOY®), whereas Replicate A employed an IgG1f allotypic variant of ipilimumab. Both allotypes are functionally equivalent in the experiments herein.

FIGS. 1A-1C show longitudinal tracking of FACS-sorted Nef RM9-specific CD8⁺ CD3⁺ lymphocytes in whole blood obtained from Mafa-A1*063+ Mauritian cynomolgus macaques treated with the indicated amounts (10 mg/kg or 1 mg/kg) of the indicated antibodies, or with vehicle. The animals had also been treated with two recombinant Ad5 vectors, one expressing the SIV Nef protein and the other expressing the SIV Gag protein, as described in greater detail at Example 1. Nef RM9⁺ cells were selected based on their binding to RM9 peptide-loaded MHC class I tetramers. “Inert” anti-CTLA-4 refers to an N297A heavy chain sequence variant that removes the site for N-linked glycosylation, generating a nonglycosylated Fc region lacking effector function. In this figure and every other figure reporting use of “inert” anti-CTLA-4 antibody the antibody was administered at 10 mg/kg. In all of FIGS. 1A-1C, 10 mg/kg anti-CTLA4-NF (upward pointing triangles) is the uppermost curve.

FIGS. 2A-2C show longitudinal tracking of FACS-sorted Gag GW9-specific CD8⁺ CD3⁺ lymphocytes in whole blood obtained from Mafa-A1*063+ Mauritian cynomolgus macaques treated with the indicated amounts (10 mg/kg or 1 mg/kg) of the indicated antibodies, or with vehicle. The animals had also been treated with two recombinant Ad5 vectors, one expressing the SIV Nef protein and the other expressing the SIV Gag protein, as described in greater detail at Example 1. Gag GW9⁺ cells were selected based on their binding to GW9 peptide-loaded MHC class I tetramers. In all of FIGS. 2A-2C, 10 mg/kg anti-CTLA4-NF (upward pointing triangles) is the uppermost curve.

FIGS. 3A-3C show longitudinal tracking of FACS-sorted Nef LT9-specific CD8⁺ CD3⁺ lymphocytes in whole blood obtained from Mafa-A1*063+ Mauritian cynomolgus macaques treated with the indicated amounts (10 mg/kg or 1 mg/kg) of the indicated antibodies, or with vehicle. The animals had also been treated with two recombinant Ad5 vectors, one expressing the SIV Nef protein and the other expressing the SIV Gag protein, as described in greater detail at Example 1. Nef LT9⁺ cells were selected based on their binding to LT9 peptide-loaded MHC class I tetramers. In all of FIGS. 3A-3C, 10 mg/kg anti-CTLA4-NF (upward pointing triangles) is the uppermost curve.

FIGS. 4A-4C present ELISPOT results showing Nef RM9-peptide induced IFN-γ production, presented as spot-forming cell (SFC) values after background subtraction, in Ficoll-isolated PBMC obtained from Mafa-A1*063+ Mauritian cynomolgus macaques 22 days (FIG. 4A), or 22 and 43 days (FIGS. 4B and 4C), after being treated with the indicated amounts (10 mg/kg or 1 mg/kg) of the indicated antibodies, or with vehicle. In this and all other figures herein, antibodies were administered at 10 mg/kg in cases where the dosing is not indicated. The animals had also been treated with two recombinant Ad5 vectors, one expressing the SIV Nef protein and the other expressing the SIV Gag protein, as described in greater detail at Example 1. PBMCs were stimulated for 18 hours with 10 μM Nef RM9 minimal optimal peptide.

FIGS. 5A-5C present ELISPOT results showing Gag GW9-peptide induced IFN-γ production, presented as spot-forming cell (SFC) values after background subtraction, in Ficoll-isolated PBMC obtained from Mafa-A1*063+ Mauritian cynomolgus macaques 22 days (FIG. 5A), or 22 and 43 days (FIGS. 5B and 5C), after being treated with the indicated amounts (10 mg/kg or 1 mg/kg) of the indicated antibodies, or with vehicle. The animals had also been treated with two recombinant Ad5 vectors, one expressing the SIV Nef protein and the other expressing the SIV Gag protein, as described in greater detail at Example 1. PBMCs were stimulated for 18 hours with 10 μM Gag GW9 minimal optimal peptide.

FIGS. 6A-6B present ELISPOT results showing Nef LT9-peptide induced IFN-γ production, presented as spot-forming cell (SFC) values after background subtraction, in Ficoll-isolated PBMC obtained from Mafa-A1*063+ Mauritian cynomolgus macaques 22 and 43 days after being treated with the indicated amounts (10 mg/kg or 1 mg/kg) of the indicated antibodies, or with vehicle. This experiment does not include data from Replicate A, only Replicates B and C. The animals had also been treated with two recombinant Ad5 vectors, one expressing the SIV Nef protein and the other expressing the SIV Gag protein, as described in greater detail at Example 1. PBMCs were stimulated for 18 hours with 10 μM Nef LT9 minimal optimal peptide.

FIGS. 7A-7C show longitudinal tracking of Ki-67⁺ CD4⁺ CD3⁺ lymphocytes (as measured by flow cytometry) circulating in whole blood of Mafa-A1*063+ Mauritian cynomolgus macaques treated with the indicated amounts (10 mg/kg or 1 mg/kg) of the indicated antibodies, or with vehicle. The animals had also been treated with two recombinant Ad5 vectors, one expressing the SIV Nef protein and the other expressing the SIV Gag protein, as described in greater detail at Example 1. Ki-67 is an intracellular marker of proliferation. Values presented for day 43 in FIGS. 7C and 8C appear to be anomalously high and likely represent outliers.

FIGS. 8A-8C show longitudinal tracking of Ki-67⁺ CD8⁺ CD3⁺ lymphocytes (as measured by flow cytometry) circulating in whole blood of Mafa-A1*063+ Mauritian cynomolgus macaques treated with the indicated amounts (10 mg/kg or 1 mg/kg) of the indicated antibodies, or with vehicle. The animals had also been treated with two recombinant Ad5 vectors, one expressing the SIV Nef protein and the other expressing the SIV Gag protein, as described in greater detail at Example 1. Ki-67 is an intracellular marker of proliferation. In all of FIGS. 8A-8C, 10 mg/kg anti-CTLA4-NF (upward pointing triangles) is the uppermost curve.

FIGS. 9A-9C present ELISPOT results showing Ad5 protein-induced IFN-γ production, presented as spot-forming cell (SFC) values after background subtraction, in Ficoll-isolated PBMC obtained from Mafa-A1*063+ Mauritian cynomolgus macaques 22 and 43 days after being treated with the indicated amounts (10 mg/kg or 1 mg/kg) of the indicated antibodies, or with vehicle. Antibodies were administered at 10 mg/kg in cases where the dosing is not indicated. The animals had also been treated with two recombinant Ad5 vectors, one expressing the SIV Nef protein and the other expressing the SIV Gag protein, as described in greater detail at Example 1. PBMCs were stimulated for 18 hours with 5×10⁸ heat-inactivated Ad5 virus particles.

FIG. 10 shows the effects of nonfucosylation of anti-CTLA-4 antibody ipilimumab on specific NK cell-mediated lysis of target cells. It provides a titration of ipilimumab (circle data points) and a nonfucosylated variant of ipilimumab (square data points, uppermost curve), compared with an isotype control (triangle data points, bottom curve), in an assay of the ability of cell line NK92 to induce specific lysis of activated T_{regs} from a human donor. See Example 2. Nonfucosylated Fc increases lytic activity of ipilimumab, reducing the EC₅₀ from 1.5 μg/ml to 0.0065 μg/ml.

FIG. 11 shows the frequency of Tregs in the blood of Mafa-A1*063+ Mauritian cynomolgus macaques treated with 10 mg/kg ipilimumab, 10 mg/kg ipilimumab-NF or with vehicle. Data were obtained from the monkeys of Replicate B. See Example 1. Ipilimumab data are presented as diamonds on a dashed line, which is generally the uppermost curve. Ipilimumab-NF data are presented as triangles on a solid line, which is generally the middle curve. Vehicle control data are presented as circles on a dotted line, which is generally the lowermost curve. Data points are the means of 6 animals with error bars representing one standard deviation.

### DETAILED DESCRIPTION OF THE INVENTION

### Definitions

In order that the present disclosure may be more readily understood, certain terms are first defined. As used in this application, except as otherwise expressly provided herein, each of the following terms shall have the meaning set forth below. Additional definitions are set forth throughout the application.

“Adjuvant,” as used herein, refers to an agent that is administered to a subject in conjunction with a vaccine to enhance the immune response to the vaccine compared with the immune response that would result from administration of the vaccine without the adjuvant. Adjuvants of the present invention are anti-CTLA-4 antibodies with enhanced ADCC activity.

“Administering,” “administer” or “administration” refers to the physical introduction of a composition comprising a therapeutic agent to a subject, using any of the various methods and delivery systems known to those skilled in the art. Preferred routes of administration for antibodies of the invention include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody of the invention can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

Administration of an anti-CTLA-4 antibody with enhanced ADCC “in conjunction with” a vaccine encompasses any order of administration or concurrent administration, including any dosing schedule or number of administrations, provided that the administration of the anti-CTLA-4 antibody with enhanced ADCC is intended to boost the immune response to the vaccine.

An “antibody” (Ab) shall include, without limitation, a glycoprotein immunoglobulin which binds specifically to an antigen and comprises at least two heavy chains (HC) and two light chains (LC) interconnected by disulfide bonds. Each heavy chain comprises a heavy chain variable region (abbreviated herein as V_{H}) and a heavy chain constant region. The heavy chain constant region comprises three domains, C_{H1}, C_{H2} and C_{H3}. Each light chain comprises a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

As used herein, and in accord with conventional interpretation, an antibody that is described as comprising “a” heavy chain and/or “a” light chain refers to antibodies that comprise “at least one” of the recited heavy and/or light chains, and thus will encompass antibodies having two or more heavy and/or light chains. Specifically, antibodies so described will encompass conventional antibodies having two substantially identical heavy chains and two substantially identical light chains. Antibody chains may be substantially identical but not entirely identical if they differ due to post-translational modifications, such as C-terminal cleavage of lysine residues, alternative glycosylation patterns, etc. Antibodies differing in fucosylation within the glycan, however, are not substantially identical.

Unless indicated otherwise or clear from the context, an antibody defined by its target specificity (e.g. an “anti-CTLA-4 antibody”) refers to antibodies that can bind to its human target (e.g. human CTLA-4). Such antibodies may or may not bind to CTLA-4 from other species.

The immunoglobulin may derive from any of the commonly known isotypes, including but not limited to IgA, secretory IgA, IgG and IgM. The IgG isotype may be divided in subclasses in certain species: IgG1, IgG2, IgG3 and IgG4 in humans, and IgG1, IgG2a, IgG2b and IgG3 in mice. “Isotype” refers to the antibody class (e.g., IgM or IgG1) that is encoded by the heavy chain constant region genes. “Antibody” includes, by way of example, both naturally occurring and non-naturally occurring antibodies, including allotypic variants; monoclonal and polyclonal antibodies; chimeric and humanized antibodies; human or non-human antibodies; wholly synthetic antibodies; and single chain antibodies. Unless otherwise indicated, or clear from the context, antibodies disclosed herein are human IgG1 antibodies. IgG1 constant domain sequences include, but are not limited to, IgG1 allotypic variants provided herein as the constant domain of ipilimumab (IgG1fa, residues 119-448 of SEQ ID NO: 11 and 119-447 of SEQ ID NO: 12) and IgG1za (SEQ ID NOs: 28 and 29).

An “isolated antibody” refers to an antibody that is substantially free of other antibodies having different antigenic specificities (e.g., an isolated antibody that binds specifically to CTLA-4 is substantially free of antibodies that bind specifically to antigens other than CTLA-4). An isolated antibody that binds specifically to CTLA-4 may, however, cross-react with other antigens, such as CTLA-4 molecules from different species. Moreover, an isolated antibody may be substantially free of other cellular material and/or chemicals. By comparison, an “isolated” nucleic acid refers to a nucleic acid composition of matter that is markedly different, i.e., has a distinctive chemical identity, nature and utility, from nucleic acids as they exist in nature. For example, an isolated DNA, unlike native DNA, is a free-standing portion of a native DNA and not an integral part of a larger structural complex, the chromosome, found in nature. Further, an isolated DNA, unlike native DNA, can be used as a PCR primer or a hybridization probe for, among other things, measuring gene expression and detecting biomarker genes or mutations for diagnosing disease or predicting the efficacy of a therapeutic. An isolated nucleic acid may also be purified so as to be substantially free of other cellular components or other contaminants, e.g., other cellular nucleic acids or proteins, using standard techniques well known in the art.

The term “monoclonal antibody” (“mAb”) refers to a preparation of antibody molecules of single molecular composition, i.e., antibody molecules whose primary sequences are essentially identical, and which exhibit a single binding specificity and affinity for a particular epitope. Monoclonal antibodies may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized” antibody refers to an antibody having CDR regions derived from non-human animal, e.g. rodent, immunoglobulin germ line sequences in which some, most or all of the amino acids outside the CDR domains are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “antibody fragment” refers to a portion of a whole antibody, generally including the “antigen-binding portion” (“antigen-binding fragment”) of an intact antibody which retains the ability to bind specifically to the antigen bound by the intact antibody and also retains the Fc region of an antibody mediating FcR binding capability.

“Antibody-dependent cell-mediated cytotoxicity” (“ADCC”) refers to an in vitro or in vivo cell-mediated reaction in which nonspecific cytotoxic cells that express FcRs (e.g., natural killer (NK) cells, macrophages, neutrophils and eosinophils) recognize antibody bound to a surface antigen on a target cell and subsequently cause lysis of the target cell. In principle, any effector cell with an activating FcR can be triggered to mediate ADCC.

“Enhanced ADCC” or “enhanced ADCC activity,” as used herein with reference to the anti-CTLA-4 antibodies of the present invention refer to ADCC activity levels greater than ADCC induced by unmodified ipilimumab. Ipilimumab with enhanced ADCC of the present invention, for example, is a modified form of ipilimumab that induces greater ADCC than ipilimumab with its native IgG1 constant domain. In the case of tremelimumab, the enhanced ADCC is also measured with reference to ipilimumab. “Ipilimumab,” “ipi” and YERVOY®, as used herein in the specification and figures, unless otherwise expressly indicated, refer to the antibody comprising the light chain of SEQ ID NO: 13 and the heavy chain of SEQ ID NO: 12 (lacking C-terminal lysine residue). In the context of the experiments of Replicate A only, “ipilimumab” encompasses an allotypic variant comprising the mutations D357E and L359M (IgG1f). In some embodiments, the level of enhancement in ADCC activity is measured as at least a two-fold, and optionally at least a ten-fold, reduction in the EC₅₀ for NK92 cell mediated cell lysis in the assay described at Example 2.

“Cancer” refers a broad group of various diseases characterized by the uncontrolled growth of abnormal cells in the body. Unregulated cell division and growth divide and grow results in the formation of malignant tumors or cells that invade neighboring tissues and may also metastasize to distant parts of the body through the lymphatic system or bloodstream.

A “cell surface receptor” refers to molecules and complexes of molecules capable of receiving a signal and transmitting such a signal across the plasma membrane of a cell.

“Effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and down-regulation of a cell surface receptor (e.g., the B cell receptor; BCR). Such effector functions generally require the Fc region to be combined with a binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) receptors and one inhibitory (FcγRIIB) receptor. Various properties of human FcγRs are summarized in Table 1. The majority of innate effector cell types co-express one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, the Fc region is a polypeptide comprising the constant region of an antibody excluding the first constant region immunoglobulin domain. In IgG, IgA and IgD antibody isotypes, the Fc region is composed of two identical protein fragments, derived from the second (C_{H2}) and third (C_{H3}) constant domains of the antibody's two heavy chains; IgM and IgE Fc regions contain three heavy chain constant domains (C_{H} domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 to the carboxy-terminus of the heavy chain, wherein the numbering is according to the EU index as in Kabat. The C_{H2} domain of a human IgG Fc region extends from about amino acid 231 to about amino acid 340, whereas the C_{H3} domain is positioned on C-terminal side of a C_{H2} domain in an Fc region, i.e., it extends from about amino acid 341 to about amino acid 447 of an IgG. As used herein, the Fc region may be a native sequence Fc or a variant Fc. Fc may also refer to this region in isolation or in the context of an Fc-comprising protein polypeptide such as a “binding protein comprising an Fc region,” also referred to as an “Fc fusion protein” (e.g., an antibody or immunoadhesin).

TABLE 1
Properties of Human FcγRs

| | Allelic | Affinity for | Isotype | |
|---|---|---|---|---|
| Fcγ | variants | human IgG | preference | Cellular distribution |
| FcγRI | None | High (K_{D} | IgG1 = 3 > 4 >> 2 | Monocytes, macrophages, |
| | described | ~10 nM) | | activated neutrophils, |
| | | | | dendritic cells? |
| FcγRIIA | H131 | Low to | IgG1 > 3 > 2 > 4 | Neutrophils, monocytes, |
| | | medium | | macrophages, eosinophils, |
| | R131 | Low | IgG1 > 3 > 4 > 2 | dendritic cells, platelets |
| FcγRIIIA | V158 | Medium | IgG1 = 3 >> 4 > 2 | NK cells, monocytes, |
| | F158 | Low | IgG1 = 3 >> 4 > 2 | macrophages, mast cells, |
| | | | | eosinophils, dendritic cells? |
| FcγRIIB | I232 | Low | IgG1 = 3 = 4 > 2 | B cells, monocytes, |
| | T232 | Low | IgG1 = 3 = 4 > 2 | macrophages, dendritic |
| | | | | cells, mast cells |

“Fucosylation” and “nonfucosylation,” as used herein, refer to the presence or absence of a core fucose residue on the N-linked glycan at position N297 of an antibody (EU numbering).

An “immune response” refers to a biological response within a vertebrate against foreign agents, which response protects the organism against these agents and diseases caused by them. The immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues.

An “immunomodulator” or “immunoregulator” refers to a component of a signaling pathway that may be involved in modulating, regulating, or modifying an immune response. “Modulating,” “regulating,” or “modifying” an immune response refers to any alteration in a cell of the immune system or in the activity of such cell. Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments of the disclosed invention, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

A “protein” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. The term “protein” is used interchangeable herein with “polypeptide.”

A “subject” includes any human or non-human animal. The term “non-human animal” includes, but is not limited to, vertebrates such as nonhuman primates, sheep, dogs, rabbits, rodents such as mice, rats and guinea pigs, avian species such as chickens, amphibians, and reptiles. In preferred embodiments, the subject is a mammal such as a nonhuman primate, sheep, dog, cat, rabbit, ferret or rodent. In more preferred embodiments of any aspect of the disclosed invention, the subject is a human. The terms, “subject” and “patient” are used interchangeably herein.

A “therapeutically effective amount” or “therapeutically effective dosage” of a drug or therapeutic agent, such as an Fc fusion protein of the invention, is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug preferably inhibits cell growth or tumor growth by at least about 20%, more preferably by at least about 40%, even more preferably by at least about 60%, and still more preferably by at least about 80% relative to untreated subjects. In the most preferred embodiments, a therapeutically effective amount or dosage of the drug completely inhibits cell growth or tumor growth, i.e., preferably inhibits cell growth or tumor growth by 100%. The ability of a compound to inhibit tumor growth can be evaluated in an animal model system, such as the CT26 colon adenocarcinoma, MC38 colon adenocarcinoma and Sa1N fibrosarcoma mouse tumor models, which are predictive of efficacy in human tumors. Alternatively, this property of a composition can be evaluated by examining the ability of the compound to inhibit cell growth, such inhibition can be measured in vitro by assays known to the skilled practitioner. In other preferred embodiments of the invention, tumor regression may be observed and continue for a period of at least about 20 days, more preferably at least about 40 days, or even more preferably at least about 60 days.

“Treatment” or “therapy” of a subject refers to any type of intervention or process performed on, or administering an active agent to, the subject with the objective of reversing, alleviating, ameliorating, inhibiting, slowing down or prevent the onset, progression, development, severity or recurrence of a symptom, complication, condition or biochemical indicia associated with a disease.

Anti-CTLA-4 Antibodies with Enhanced ADCC are More Effective as Adjuvants

It is now recognized that CTLA-4 exerts its physiological function primarily through two distinct effects on the two major subsets of CD4⁺ T cells: (1) down-modulation of helper T cell activity, and (2) enhancement of the immunosuppressive activity of regulatory T cells (T_{regs}). Lenschow et al. (1996) Ann. Rev. Immunol. 14:233; Wing et al. (2008) Science 322:271; Peggs et al. (2009) J Exp. Med. 206:1717. T_{regs} are known to constitutively express high levels of surface CTLA-4, and it has been suggested that this molecule is integral to their regulatory function. Takahashi et al. (2000) J. Exp. Med. 192:303; Birebent et al. (2004) Eur. J. Immunol. 34:3485. Accordingly, the T_{reg} population may be most susceptible to the effects of CTLA-4 blockade. Studies of ipilimumab patients also show that responders, as distinguished from non-responders, exhibit decreased T_{reg} infiltration after treatment, with depletion occurring via an ADCC mechanism and mediated by FcγRIIIA-expressing non-classical (CD14⁺ CD16⁺⁺) monocytes. Romano et al. (2014) J. Immunotherapy of Cancer 2(Suppl. 3):O14.

In one aspect, the present invention provides improved methods of enhancing the immune response to vaccines by administering anti-CTLA-4 antibodies, such as ipilimumab, modified to exhibit enhanced ADCC. Such antibodies exhibit improved vaccine adjuvant activity in light of the experimental results provided herein.

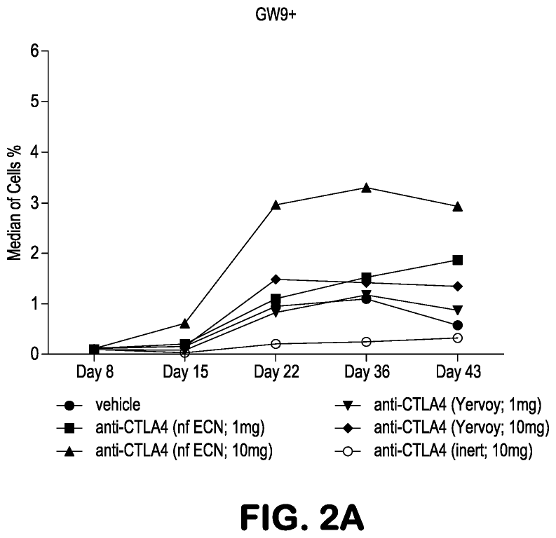

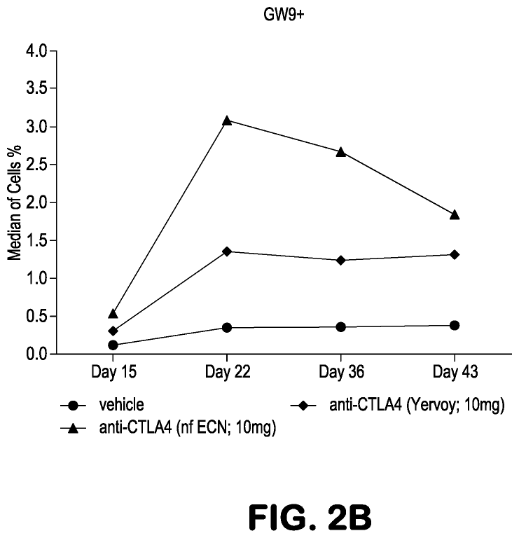

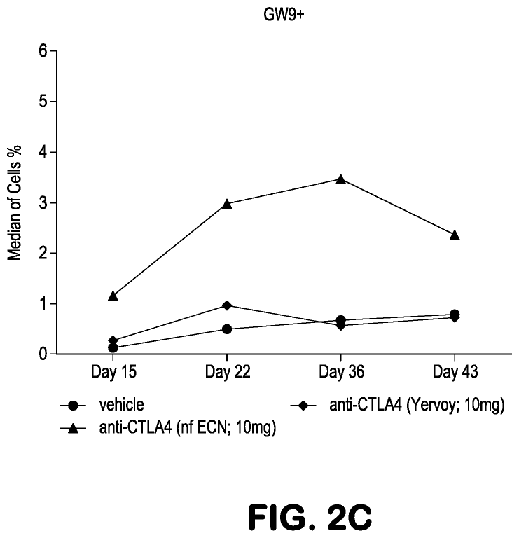

Anti-CTLA-4 antibodies with enhanced ADCC activity would not have been expected to enhance immune response to a vaccine. Prior experiments on the effects of such antibodies in treating cancer had shown, in fact, that treatment with an anti-CTLA4 antibody, with or without enhanced ADCC, actually increased the population of regulatory T cells (T_{regs}) in the periphery (i.e. outside the tumor microenvironment), which would be expected to reduce vaccine response rather than enhance it. See Selby et al. (2013) Cancer Immunol. Res. 1:32, at Abstract, and FIG. 2A.

Use of such anti-CTLA-4 antibodies with enhanced ADCC may enhance vaccine efficacy at a given dose of vaccine, or may allow for lower dosing to attain any given level of efficacy, and/or may increase the persistence of immune response. The methods of the present invention, involving use of anti-CTLA-4 antibodies with enhanced ADCC activity, would be expected to enhance both B cell and T cell immune responses, and against both self and foreign antigens, and against both dominant and subdominant epitopes. As such, the methods of the present invention may enhance the effectiveness of prophylactic vaccines in subjects naïve to the vaccine antigen, and also may enhance the effectiveness of therapeutic vaccines in subjects in which a pre-existing (prior to vaccination) anti-antigen immune response has become exhausted.

### Mouse Model Experiments

The OVA vaccine prime-boost model was used to test the effects of enhanced ADCC activity on the adjuvant activity of anti-CTLA-4 antibodies. Mice were treated with anti-mouse CTLA-4 antibody 9D9 as either a mouse IgG1, IgG2b, or IgG1-D265A (which results in very poor Fc-associated effector functions—Baudino et al. (2008) J. Immunol. 181:6664), or as a mouse IgG2a (which exhibits enhanced ADCC). See WO 2014/089113. Experiments also included a mIgG2a isotype control, OVA-only and naïve mice. Mouse IgG2a antibodies exhibit enhanced ADCC compared with the human IgG1 antibody ipilimumab.

Mice were immunized with OVA peptide subcutaneously (sc) on day 0 and challenged with OVA peptide sc at day 14. Antibodies were dosed at 0.1 mg/dose intraperitoneally (ip) on days −1, 1, 13 and 15, with 10 mice per group. At day 21, mice were bled for anti-OVA titers in serum and blood, and for assays.

Spleens in mice treated with mIgG2a anti-CTLA-4 mAb (which has enhanced ADCC) were typically ˜20% heavier than other groups, which were all similar to each other. These same mice exhibited enhanced serum anti-OVA IgG titers at day 21, as well as enhanced OVA-specific IFN-γ production in the spleen as measure in by ELISPOT.

Other experiments demonstrated that there was no enhancement of depletion of Foxp3⁺ T_{regs} (measured as a percentage of CD45⁺ cells) in the blood, spleen or inguinal lymph nodes of mice treated with 9D9 IgG2a with enhanced ADCC activity as compared to other isotypes.

These same antibodies were also tested in the myelin oligodendrocyte glycoprotein (MOG) peptide-induced experimental autoimmune encephalomyelitis (EAE) model. MOG₃₅₋₅₅/CFA was administered to 63 female C57BL/6 mice (5 mice/group+3 naïve mice) sc on day 0. Pertussis toxin was administered iv on days 0 and 2. Antibodies (9D9 IgG1-D265A, 9D9 IgG2a, mIgG1 isotype control, and non-blocking anti-mCTLA-4 mAb 5G6-mIgG2a) were administered on days 0, 3 and 6, with the day 0 antibody dose administered in between the MOG and pertussis toxin. Mice were sacrificed on day 15. Both mIgG2a antibodies enhanced EAE disease scores dramatically compared to isotype control, with mIgG1-D265A providing a more modest enhancement. Enhanced disease score in this model correlates with enhanced anti-MOG immune response, and thus enhance adjuvant activity. As with the OVA model above, enhanced ADCC mAbs (mIgG2a) do not deplete Foxp3⁺ T_{regs} (measured as a percentage of CD45⁺ cells) in the spleen or lymph nodes, and also not in the central nervous system (CNS).

In both OVA- and MOG-induced immune response models, anti-CTLA-4 mAbs with enhanced ADCC activity (mIgG2a), both blocking antibodies and non-blocking antibodies, elicit greater immune responses, but do not cause T_{reg} depletion.

### Cynomolgus Monkey Experiments

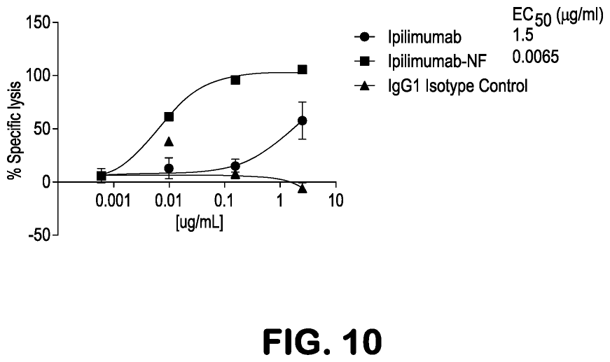

Additional experiments, as disclosed herein, investigated the role of enhanced ADCC activity on the adjuvant activity of anti-CTLA-4 antibodies in primates (cynomolgus monkeys) using anti-human CTLA-4 antibody ipilimumab (YERVOY®) and nonfucosylated ipilimumab (ipi-NF), which has enhanced ADCC (FIG. 10).

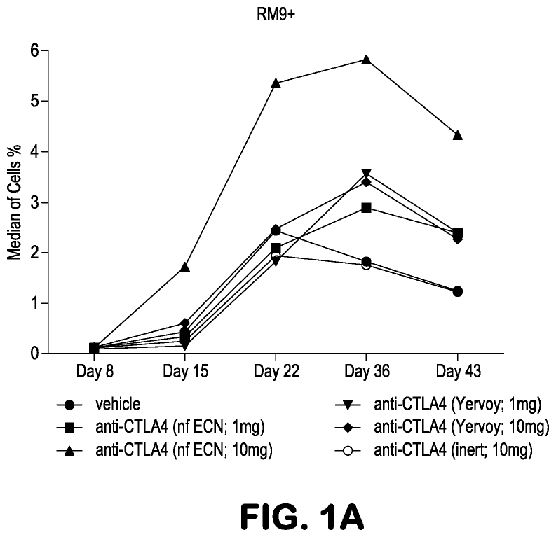

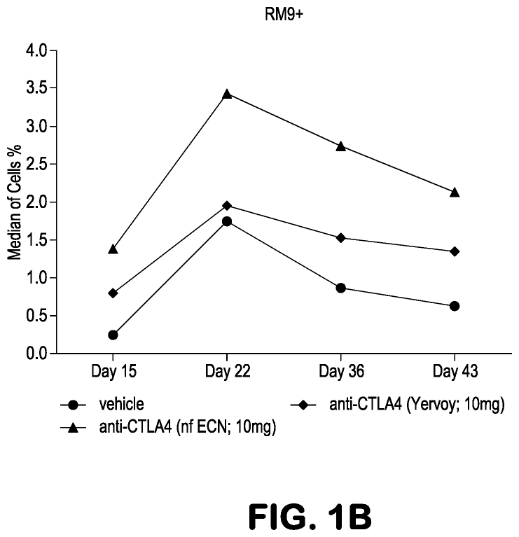

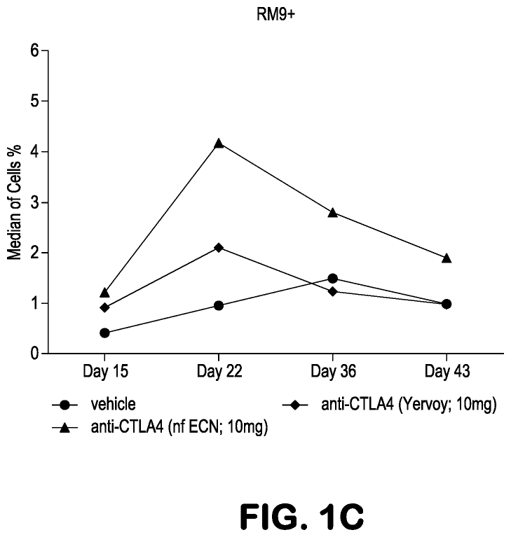

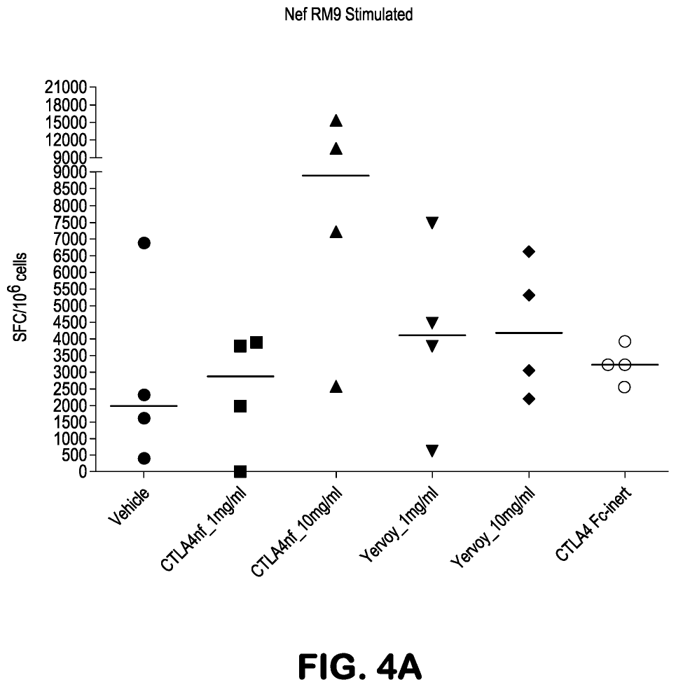

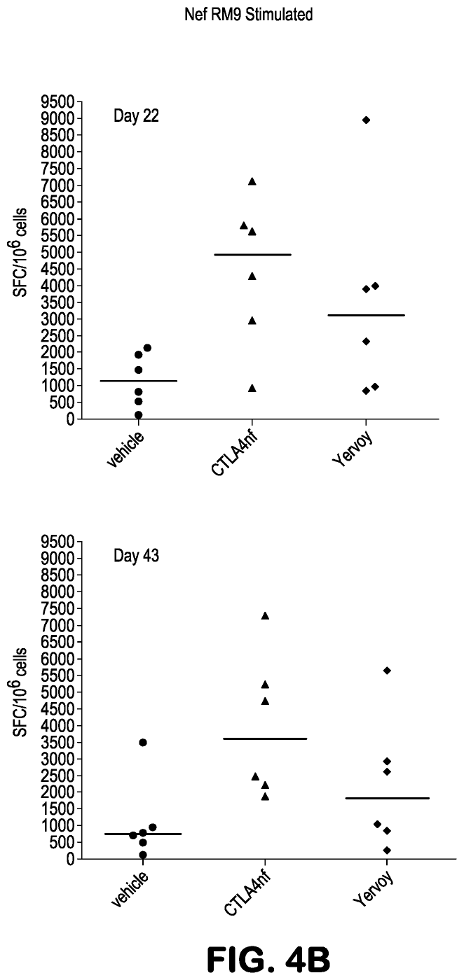

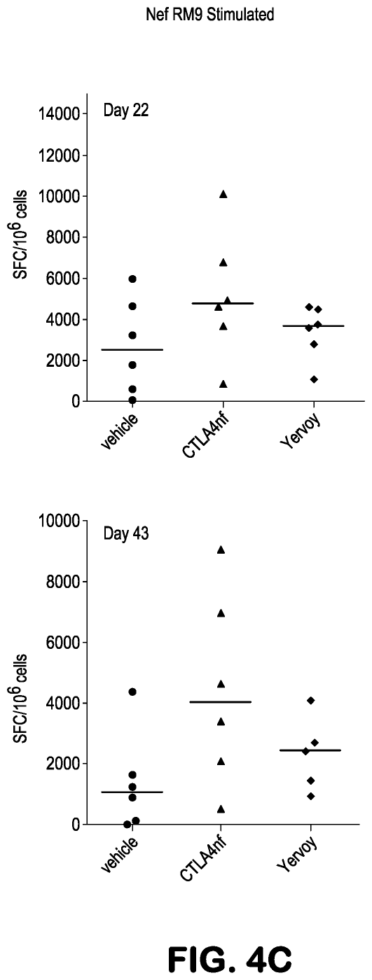

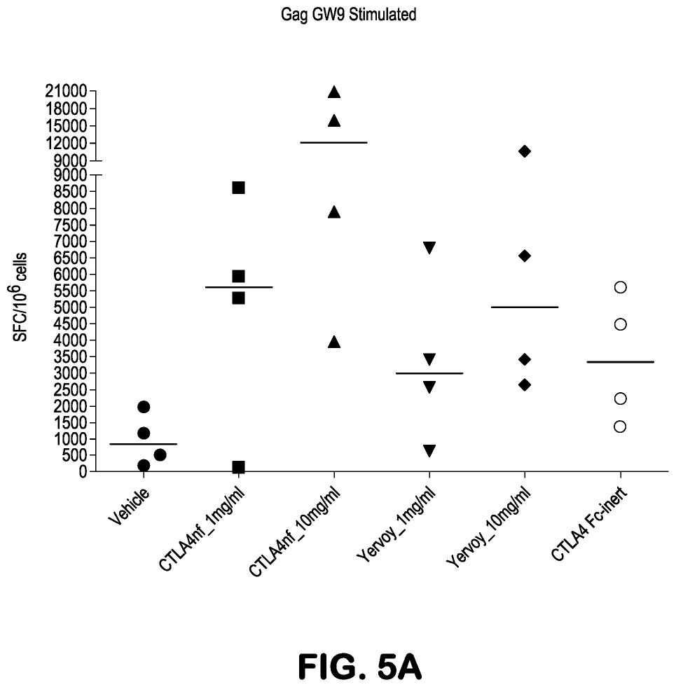

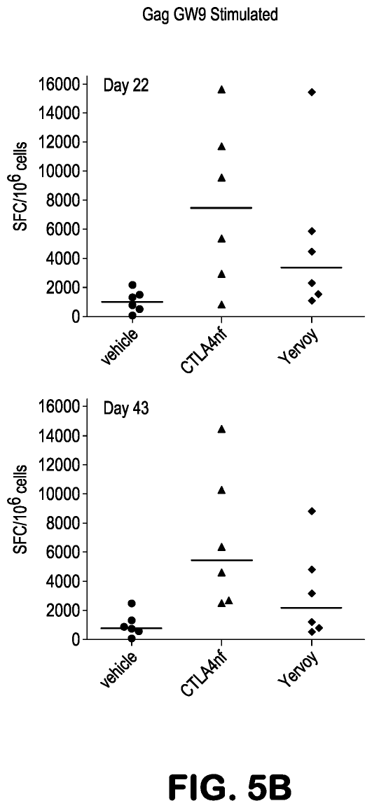

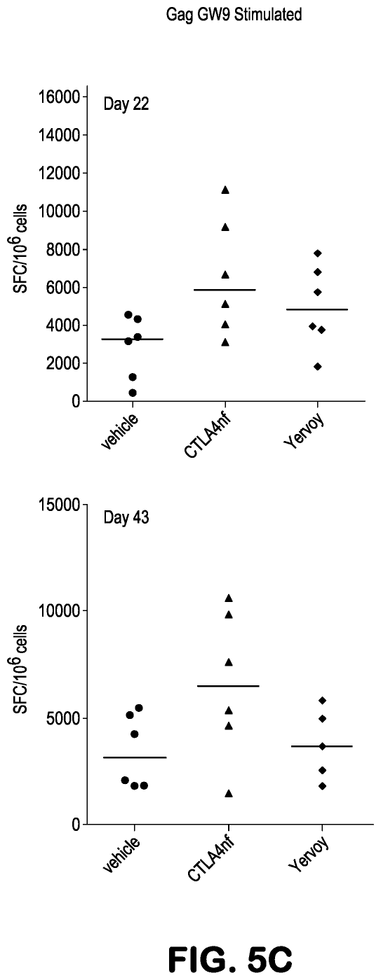

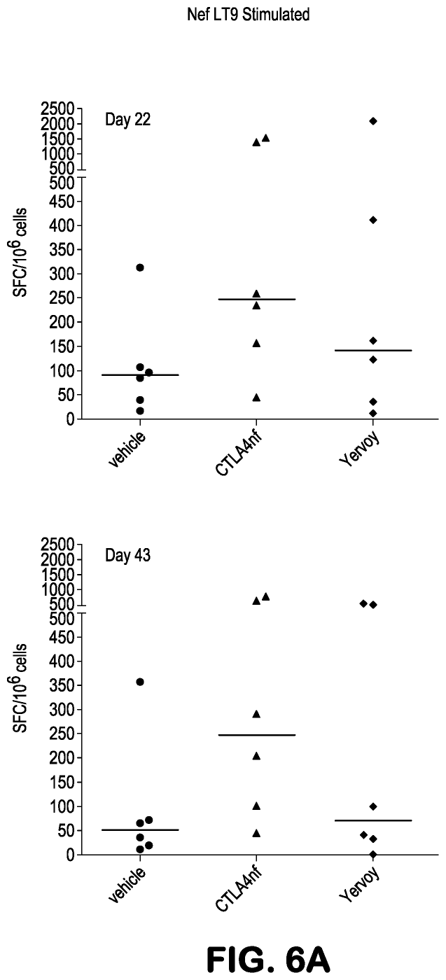

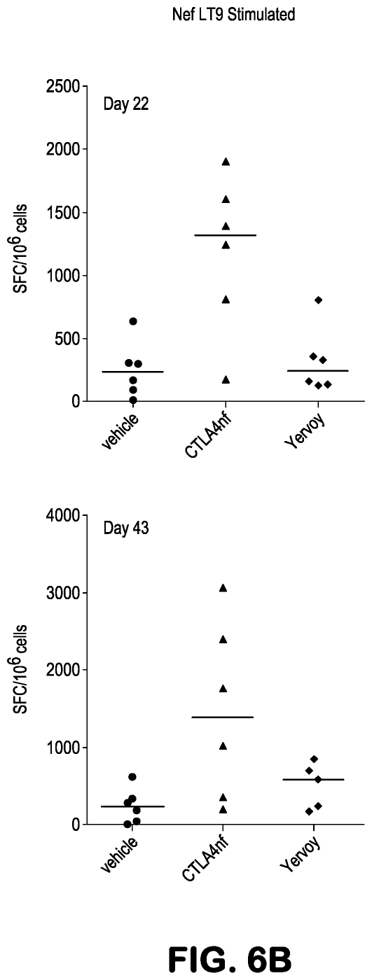

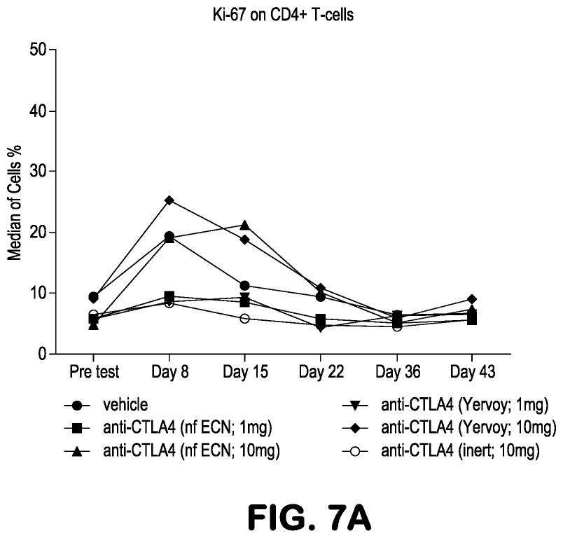

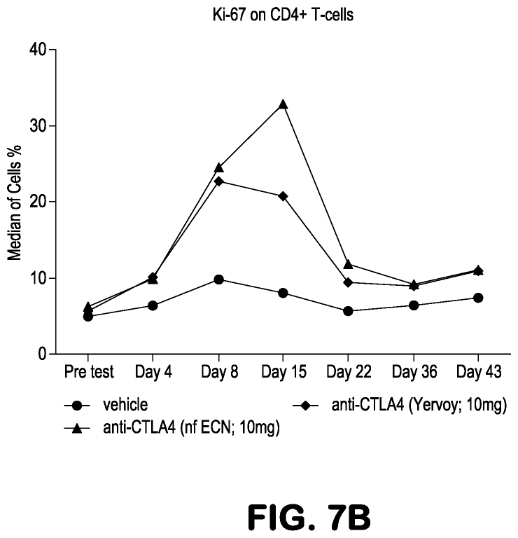

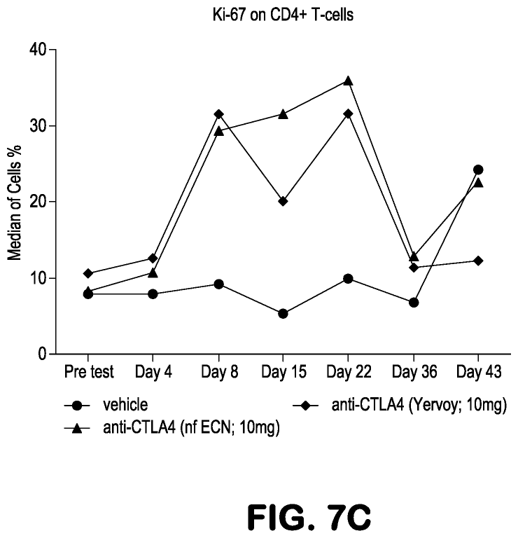

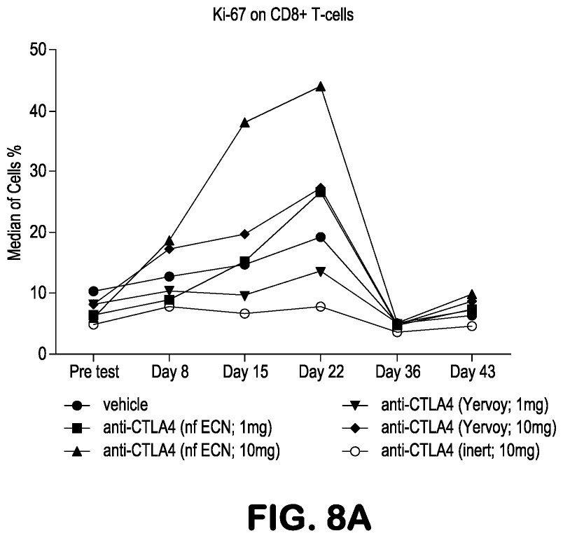

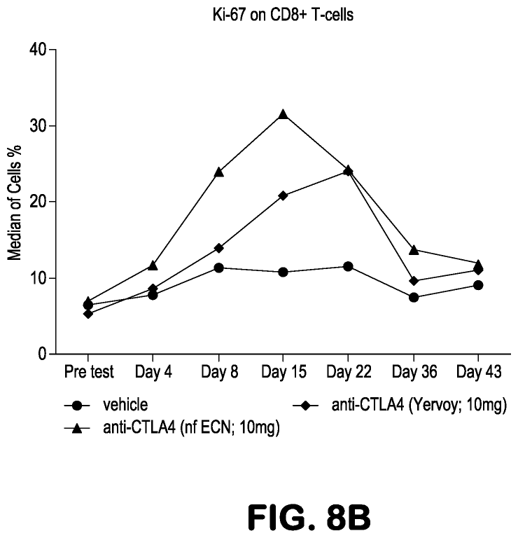

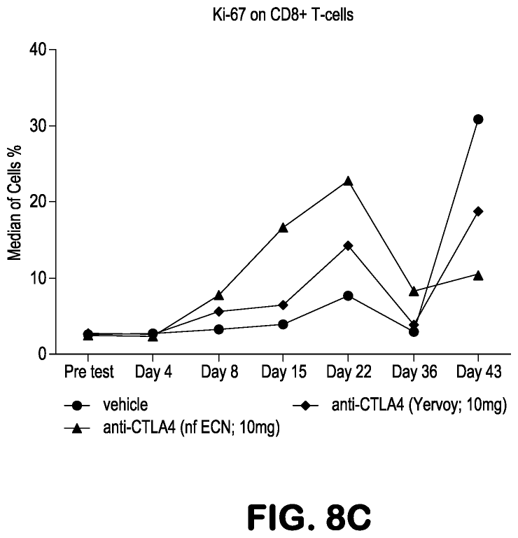

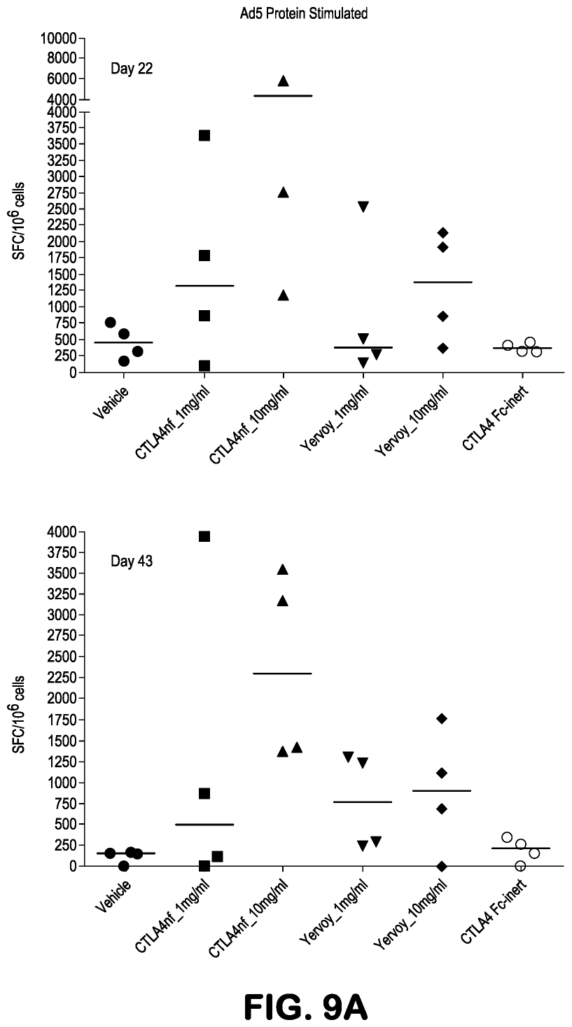

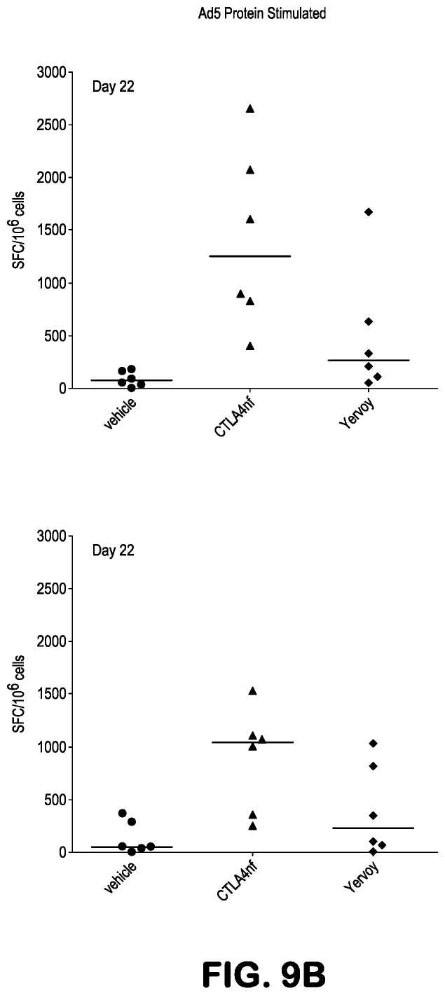

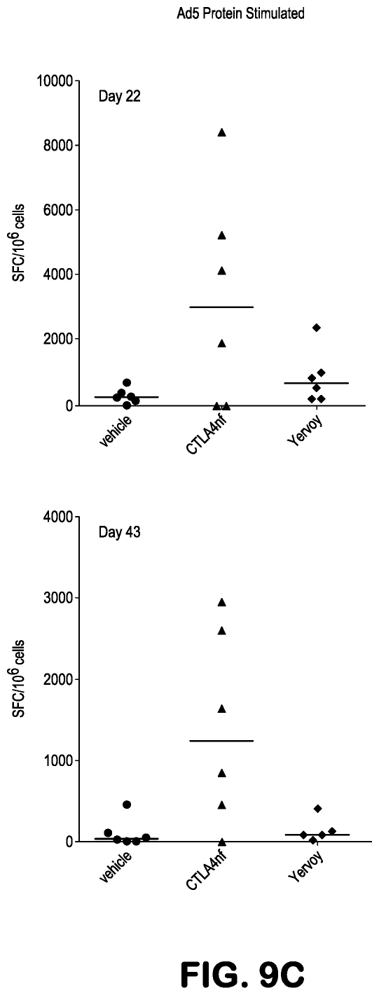

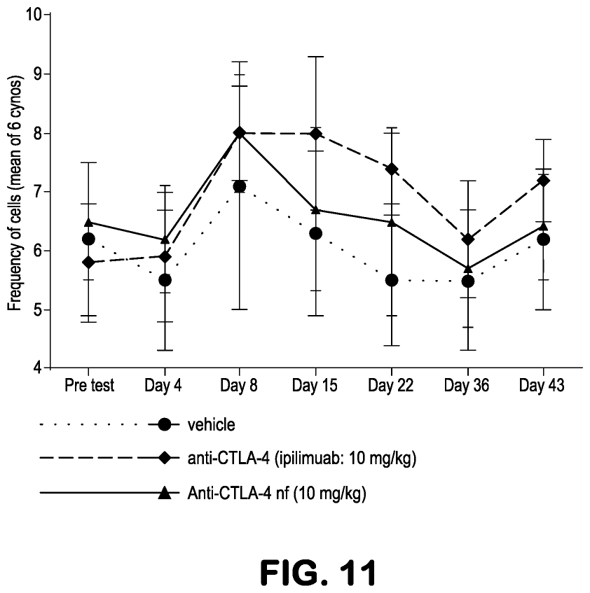

As disclosed in the various figures and examples, and consistent with the mouse results, anti-CTLA-4 antibodies with enhanced ADCC elicited greater and more robust immune responses than otherwise equivalent anti-CTLA-4 antibodies without enhanced ADCC, i.e. ipilimumab-NF versus ipilimumab. This enhanced immune response was reflected in vaccine antigen-specific CD8⁺ T cell responses (FIGS. 1A-1C, 2A-2C and 3A-3C), vaccine antigen-induced IFN-γ production (FIGS. 4A-4C, 5A-5C and 6A-6B) and Ad5-induced IFN-γ production (FIGS. 9A-9C). Ipilimumab-NF also increased CD4⁺ and CD8⁺ T cell proliferation as measured by Ki-67 expression (FIGS. 7A-7C and 8A-8C). The enhanced ADCC form of ipilimumab (ipilimumab-NF) did not cause T_{reg} depletion in the blood of the monkeys being studied (FIG. 11).

Improved Anti-CTLA-4 Antibodies with Enhanced Effector Functions

Various modifications to the Fc region of antibodies have been shown to enhance effector function. In mice, enhanced binding to activating Fc gamma receptors and reduced binding to the Fc gamma inhibitory receptor follow the hierarchy: mIgG2a>>mIgG2b>>mIgG1D265A. This hierarchy follows the activity ratio of the binding of immunoglobulin Fc regions to activating Fc receptors versus inhibitory Fc receptors (known as the A/I ration) defined by Nimmerjahn & Ravetch (2005) Science 310:1510 and determined for antibodies mediating ADCC function.

In certain aspects the improved anti-CTLA-4 antibody of the present invention is a human IgG1 antibody. ADCC activity in the anti-CTLA-4 antibodies of the present invention may be enhanced, e.g., by introducing one or more amino acid substitutions in the Fc region, altering the glycosylation pattern at the N-linked glycan, or both.

Fc Mutations that Enhance Effector Function

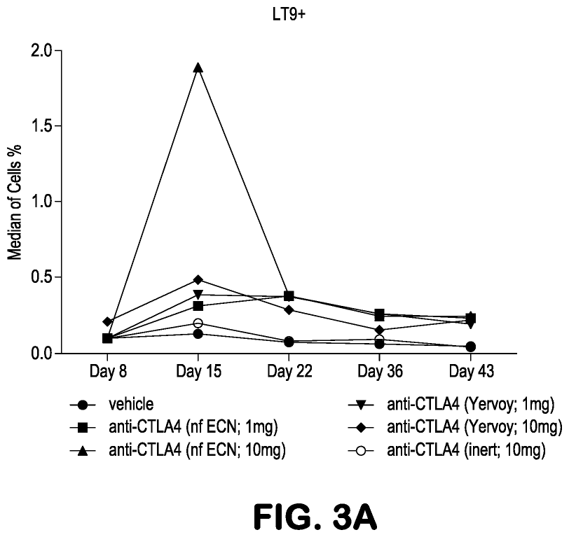

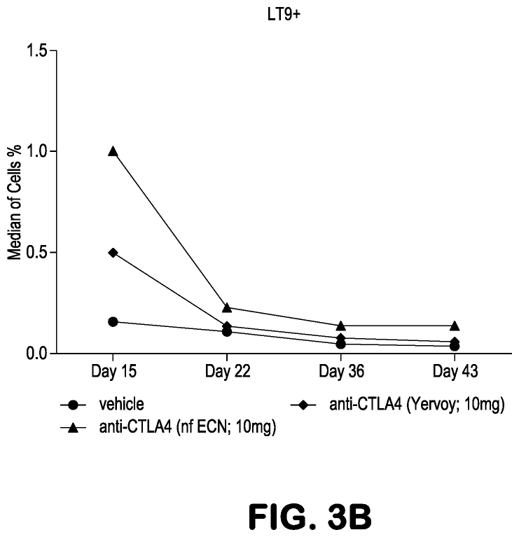

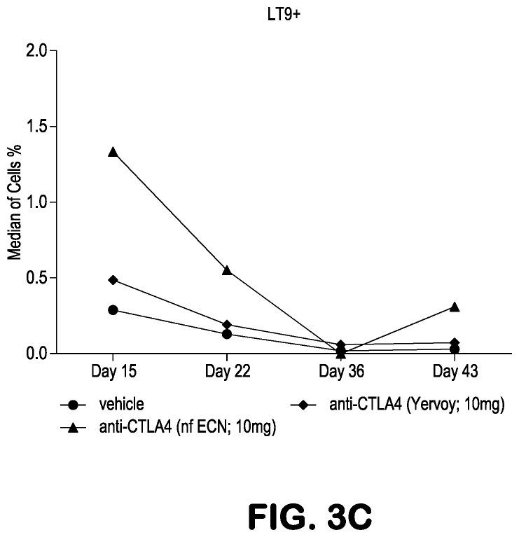

In some embodiments, ADCC activity is increased by modifying the amino acid sequence of the Fc region, e.g. adding mutations to a naturally occurring human IgG1 sequence to enhance ADCC. With regard to ADCC activity, human IgG1≥IgG3>>IgG4≥IgG2, so an IgG1 constant domain, rather than an IgG2 or IgG4, might be chosen as a starting point from which to enhance ADCC. As defined herein, unmodified human IgG1 as it occurs in ipilimumab does not have enhanced ADCC. The Fc region may be modified to increase antibody dependent cellular cytotoxicity (ADCC) and/or to increase the affinity for an Fcγ receptor (FcγR) by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. See WO 2012/142515; see also WO 00/42072. Exemplary individual substitutions include 236A, 239D, 239E, 268D, 267E, 268E, 268F, 324T, 332D, and 332E. Exemplary clusters of variants include 239D/332E, 236A/332E, 236A/239D/332E, 268F/324T, 267E/268F, 267E/324T, and 267E/268F/324T. For example, human IgG1Fcs comprising the G236A variant, which can optionally be combined with I332E, have been shown to increase the FcγIIA/FcγIIB binding affinity ratio approximately 15-fold. Richards et al. (2008) Mol. Cancer Therap. 7:2517; Moore et al. (2010) mAbs 2:181. Other modifications for enhancing FcγR and complement interactions include but are not limited to substitutions 298A, 333A, 334A, 326A, 247I, 339D, 339Q, 280H, 290S, 298D, 298V, 243L, 292P, 300L, 396L, 305I, and 396L. These and other modifications are reviewed in Strohl (2009) Current Opinion in Biotechnology 20:685-691. Specifically, both ADCC and CDC may be enhanced by changes at position E333 of IgG1, e.g. E333A. Shields et al. (2001) J Biol. Chem. 276:6591. The use of P2471 and A339D/Q mutations to enhance effector function in an IgG1 is disclosed at WO 2006/020114, and D280H, K290S±S298D/V is disclosed at WO 2004/074455. The K326A/W and E333A/S variants have been shown to increase effector function in human IgG1, and E333S in IgG2. Idusogie et al (2001) J. Immunol. 166:2571. Other experiments have shown that G236A/S239D/A330L/I332E results in enhanced binding to FcRIIa and FcRIIIa. Smith et al. (2012) Proc. Nat'l Acad. Sci. (USA) 109:6181; Boumazos et al. (2014) Cell 158:1243. Unless otherwise indicated, or clear from the context, amino acid residue numbering in the Fc region of an antibody is according to the EU numbering convention (the EU index as in Kabat et al. (1991) Sequences of Proteins of Immunological Interest, National Institutes of Health, Bethesda, Md.; see also FIGS. 3c-3f of U.S. Pat. App. Pub. No. 2008/0248028), except when specifically referring to residues in a sequence in the Sequence Listing, in which case numbering is necessarily consecutive. For example, literature references regarding the effects of amino acid substitutions in the Fc region will typically use EU numbering, which allows for reference to any given residue in the Fc region of an antibody by the same number regardless of the length of the variable domain to which is it attached. In rare cases it may be necessary to refer to the document being referenced to confirm the precise Fc residue being referred to.

Specifically, the binding sites on human IgG1 for FcγRI, FcγRII, FcγRIII and FcRn have been mapped, and variants with improved binding have been described. Shields et al. (2001) J. Biol. Chem. 276:6591-6604. Specific mutations at positions 256, 290, 298, 333, 334 and 339 were shown to improve binding to FcγRIII, including the combination mutants T256A/S298A, S298A/E333A, S298A/K224A and S298A/E333A/K334A (having enhanced FcγRIIIa binding and ADCC activity). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys. Lazar et al. (2006) Proc. Nat'l Acad. Sci. (USA) 103:4005; Awan et al. (2010) Blood 115:1204; Desjarlais & Lazar (2011) Exp. Cell Res. 317:1278. Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in macaques. Lazar et al. (2006) Proc. Nat'l Acad. Sci. (USA) 103:4005. In addition, IgG1 mutants containing L235V, F243L, R292P, Y300L, V305I and P396L mutations which exhibited enhanced binding to FcγRIIIa and concomitantly enhanced ADCC activity in transgenic mice expressing human FcγRIIIa in models of B cell malignancies and breast cancer have been identified. Stavenhagen et al. (2007) Cancer Res. 67:8882; U.S. Pat. No. 8,652,466; Nordstrom et al. (2011) Breast Cancer Res. 13:R123.

Different IgG isotypes also exhibit differential CDC activity (IgG3>IgG1>>IgG2≈IgG4). Dangl et al. (1988) EMBO J. 7:1989. For uses in which enhanced CDC is desired, it is also possible to introduce mutations that increase binding to C1q. The ability to recruit complement (CDC) may be enhanced by mutations at K326 and/or E333 in an IgG2, such as K326W (which reduces ADCC activity) and E333S, to increase binding to C1q, the first component of the complement cascade. Idusogie et al. (2001) J. Immunol. 166:2571. Introduction of S267E/H268F/S324T (alone or in any combination) into human IgG1 enhances C1q binding. Moore et al. (2010) mAbs 2:181. The Fc region of the IgG1/IgG3 hybrid isotype antibody “113F” of Natsume et al. (2008) Cancer Res. 68:3863 (FIG. 1 therein) also confers enhanced CDC. See also Michaelsen et al. (2009) Scand. J. Immunol. 70:553 and Redpath et al. (1998) Immunology 93:595.

Additional mutations that can increase or decrease effector function are disclosed at Dall'Acqua et al. (2006) J. Immunol. 177:1129. See also Carter (2006) Nat. Rev. Immunol. 6:343; Presta (2008) Curr. Op. Immunol. 20:460.

In some embodiments, amino add substitutions in the Fc region to enhance ADCC may be made in various IgG1 allotypes, including but not limited to the IgG1fa allotype of ipilimumab (residues 119-448 of SEQ ID NO: 11 and 119-447 of SEQ ID NO: 12) and IgG1za (SEQ ID NOs: 28 and 29).

Nonfucosylated Anti-CTLA-4 Antibodies with Enhanced ADCC

Experiments comparing nonfucosylated otherwise unmodified IgG1f antibodies show enhanced binding to activating Fcγ receptors, as shown in Table 2, demonstrating their suitability for use in the enhanced anti-CTLA-4 antibodies of the present invention. Allotype IgG1f has D357E and L359M mutations relative to ipilimumab allotype IgG1fa (SEQ ID NOs: 11 and 12). IgG1f has K97R, D239E and L241M mutations relative to allotype IgG1za (SEQ ID NOs: 28 and 29), which are equivalent to K215R, D357E and L359M mutations relative to ipilimumab sequence numbering of SEQ ID NOs: 11 and 12.

TABLE 2
Fc Receptor Binding of Nonfucosylated
IgG1f Fc Regions Kd Values (nM)

| Fcγ Receptor | IgG1f | IgG1f-NF |
|---|---|---|
| CD16-V158 (FcγRIIIa) | 97 | 11 |
| CD32-H131 (FcγRIIa) | 530 | 560 |
| CD32-R131 (FcγRIIa) | 960 | 710 |
| CD32B (FcγRIIb) | — | — |
| CD64 (FcγRIa) | 0.2 | 0.1 |

### Reduced Fucosylation, Nonfucosylation and Hypofucosylation

The interaction of antibodies with FcγRs can also be enhanced by modifying the glycan moiety attached to each Fc fragment at the N297 residue. In particular, the absence of core fucose residues strongly enhances ADCC via improved binding of IgG to activating FcγRIIIA without altering antigen binding or CDC. Natsume et al. (2009) Drug Des. Devel. Ther. 3:7. There is convincing evidence that afucosylated tumor-specific antibodies translate into enhanced therapeutic activity in mouse models in vivo. Nimmerjahn & Ravetch (2005) Science 310:1510; Mossner et al. (2010) Blood 115:4393.

Modification of antibody glycosylation can be accomplished by, for example, expressing the antibody in a host cell with altered glycosylation machinery. Antibodies with reduced or eliminated fucosylation, which exhibit enhanced ADCC, are particularly useful in the methods of the present invention. Cells with altered glycosylation machinery have been described in the art and can be used as host cells in which to express recombinant antibodies of this disclosure to thereby produce an antibody with altered glycosylation. For example, the cell lines Ms704, Ms705, and Ms709 lack the fucosyltransferase gene, FUT8 (α-(1,6) fucosyltransferase (see U.S. Pat. App. Publication No. 20040110704; Yamane-Ohnuki et al. (2004) Biotechnol. Bioeng. 87: 614), such that antibodies expressed in these cell lines lack fucose on their carbohydrates. As another example, EP 1176195 also describes a cell line with a functionally disrupted FUT8 gene as well as cell lines that have little or no activity for adding fucose to the N-acetylglucosamine that binds to the Fc region of the antibody, for example, the rat myeloma cell line YB2/0 (ATCC CRL 1662). PCT Publication WO 03/035835 describes a variant CHO cell line, Lec13, with reduced ability to attach fucose to Asn(297)-linked carbohydrates, also resulting in hypofucosylation of antibodies expressed in that host cell. See also Shields et al. (2002) J. Biol. Chem. 277:26733. Antibodies with a modified glycosylation profile can also be produced in chicken eggs, as described in PCT Publication No. WO 2006/089231. Alternatively, antibodies with a modified glycosylation profile can be produced in plant cells, such as Lemna. See e.g. U.S. Publication No. 2012/0276086. PCT Publication No. WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies. See also Umaña et al. (1999) Nat. Biotech. 17:176. Alternatively, the fucose residues of the antibody may be cleaved off using a fucosidase enzyme. For example, the enzyme alpha-L-fucosidase removes fucosyl residues from antibodies. Tarentino et al. (1975) Biochem. 14:5516. Antibodies with reduced fucosylation may also be produced in cells harboring a recombinant gene encoding an enzyme that uses GDP-6-deoxy-D-lyxo-4-hexylose as a substrate, such as GDP-6-deoxy-D-lyxo-4-hexylose reductase (RMD), as described at U.S. Pat. No. 8,642,292. Alternatively, cells may be grown in medium containing fucose analogs that block the addition of fucose residues to the N-linked glycan or a glycoprotein, such as antibody, produced by cells grown in the medium. U.S. Pat. No. 8,163,551; WO 09/135181.

Because nonfucosylated antibodies exhibit greatly enhanced ADCC compared with fucosylated antibodies, antibody preparations need not be completely free of fucosylated heavy chains to be useful in the methods of the present invention. Residual levels of fucosylated heavy chains will not significantly interfere with the ADCC activity of a preparation substantially of nonfucosylated heavy chains. Antibodies produced in conventional CHO cells, which are fully competent to add core fucose to N-glycans, may nevertheless comprise from a few percent up to 15% nonfucosylated antibodies. Nonfucosylated antibodies may exhibit ten-fold higher affinity for CD16, and up to 30- to 100-fold enhancement of ADCC activity, so even a small increase in the proportion of nonfucosylated antibodies may drastically increase the ADCC activity of a preparation. Any preparation comprising more nonfucosylated antibodies than would be produced in normal CHO cells in culture may exhibit some level of enhanced ADCC. Such antibody preparations are referred to herein as preparations having reduced fucosylation. Depending on the original level of nonfucosylation obtained from normal CHO cells, reduced fucosylation preparations may comprise as little as 50%, 30%, 20%, 10% and even 5% nonfucosylated antibodies. Reduced fucosylation is functionally defined as preparations exhibiting two-fold or greater enhancement of ADCC compared with antibodies prepared in normal CHO cells, and not with reference to any fixed percentage of nonfucosylated species.

In other embodiments the level of nonfucosylation is structurally defined. As used herein, nonfucosylated or afucosylated (terms used synonymously) antibody preparations are antibody preparations comprising greater than 95% nonfucosylated antibody heavy chains, including 100%. Hypofucosylated antibody preparations are antibody preparations comprising less than or equal to 95% heavy chains lacking fucose, e.g. antibody preparations in which between 80 and 95% of heavy chains lack fucose, such as between 85 and 95%, and between 90 and 95%. Unless otherwise indicated, hypofucosylated refers to antibody preparations in which 80 to 95% of heavy chains lack fucose, nonfucosylated refers to antibody preparations in which over 95% of heavy chains lack fucose, and “hypofucosylated or nonfucosylated” refers to antibody preparations in which 80% or more of heavy chains lack fucose.

In some embodiments, hypofucosylated or nonfucosylated antibodies are produced in cells lacking an enzyme essential to fucosylation, such as FUT8 (e.g. U.S. Pat. No. 7,214,775), or in cells in which an exogenous enzyme partially depletes the pool of metabolic precursors for fucosylation (e.g. U.S. Pat. No. 8,642,292), or in cells cultured in the presence of a small molecule inhibitor of an enzyme involved in fucosylation (e.g. WO 09/135181).

The level of fucosylation in an antibody preparation may be determined by any method known in the art, including but not limited to gel electrophoresis, liquid chromatography, and mass spectrometry. Unless otherwise indicated, for the purposes of the present invention, the level of fucosylation in an antibody preparation is determined by hydrophilic interaction chromatography (or hydrophilic interaction liquid chromatography, HILIC), essentially as described at Example 3. To determine the level of fucosylation of an antibody preparation, samples are denatured treated with PNGase F to cleave N-linked glycans, which are then analyzed for fucose content. LC/MS of full-length antibody chains is an alternative method to detect the level of fucosylation of an antibody preparation, but mass spectroscopy is inherently less quantitative.

### Nonfucosylated Ipilimumab Exhibits Enhanced ADCC

The nonfucosylated form of ipilimumab was shown to be more effective at eliciting NK92 cell based lysis of activated T_{regs} from a human donor, decreasing the EC₅₀ from 1.5 μg/mL to 6.5 ng/mL. See FIG. 10.

### Additional Potential Fc Modifications

Fc regions can be mutated to increase the affinity of IgG for the neonatal Fc receptor, FcRn, which prolongs the in vivo half-life of antibodies and results in increased anti-tumor activity. For example, introduction of M428L/N434S mutations into the Fc regions of bevacizumab (VEGF-specific) and cetuximab (EGFR-specific) increased antibody half-life in monkeys and improved anti-tumor responses in mice. Zalevsky et al. (2010) Nat. Biotechnol. 28:157.

### Anti-CTLA-4 Antibodies

In certain embodiments, the starting anti-CTLA-4 antibody to be modified to enhance ADCC is ipilimumab or tremelimumab, or antibodies sharing their variable domain sequences. Monoclonal antibodies that recognize and bind to the extracellular domain of CTLA-4 are described in U.S. Pat. No. 5,977,318. Human monoclonal antibodies of this disclosure can be generated using various methods, for example, using transgenic or transchromosomic mice carrying parts of the human immune system rather than the mouse system, or using in vitro display technologies such as phage or yeast display. See e.g. Bradbury et al. (2011) Nat. Biotechnol. 29(3):245. Transgenic and transchromosomic mice include mice referred to herein as the HUMAB MOUSE® (Lonberg et al. (1994) Nature 368:856) and KM MOUSE® (WO 02/43478), respectively. The production of exemplary human anti-human CTLA-4 antibodies of this disclosure is described in detail in U.S. Pat. Nos. 6,984,720 and 7,605,238. The human IgG1 anti-CTLA-4 antibody identified as 10D1 in these patents is also known as ipilimumab (also formerly known as MDX-010 and BMS-734016), which is marketed as YERVOY®. Other exemplary human anti-CTLA-4 antibodies of this disclosure are described in U.S. Pat. Nos. 6,682,736 and 7,109,003, including tremelimumab (formerly ticilimumab; CP-675,206), a human IgG2 anti-human CTLA-4 antibody.

Ipilimumab, a human anti-human CTLA-4 monoclonal antibody, has been approved for the treatment of unresectable or metastatic melanoma and for adjuvant treatment of stage III melanoma, and is in clinical testing in other cancers, often in combination with other agents. Hoos et al. (2010) Semin. Oncol. 37:533; Hodi et al. (2010)N Engl. J. Med. 363:711; Pardoll (2012) Nat. Immunol. 13(12): 1129. Ipilimumab has a human IgG1 isotype, which binds best to most human Fc receptors (Bruhns et al. (2009) Blood 113: 3716).

In contrast, tremelimumab is an IgG2 isotype, which does not bind efficiently to Fc receptors, except for the FcγRIIa variant H131. Bruhns et al. (2009) Blood 113:3716. Tremelimumab is an IgG2 isotype and thus exhibits lower ADCC than ipilimumab, which is an IgG1. Converting tremelimumab to an IgG1, by replacing the heavy chain constant domain to create “treme-IgG1,” would be expected to increase ADCC to a level similar to ipilimumab. In some embodiments, the methods of the present invention involve use of variants of tremelimumab or treme-IgG1 having ADCC greater than ipilimumab as vaccine adjuvants.

Additional anti-CTLA-4 Antibodies

Additional anti-CTLA-4 antibody-related inventions are disclosed in the following commonly-assigned patent application publications, the disclosures of which are hereby incorporated by reference in their entireties: WO 1993/000431; WO 97/020574; WO 00/032231; WO 2001/014424; WO 2003/086459; WO 2005/003298; WO 2006/121168; WO 2007/056540; WO 2007/067959; WO 2008/109075; WO 2009/148915; WO 2010/014784; WO 2011/011027; WO 2010/042433; WO 2011/146382; WO 2012/027536; WO 2013/138702; WO 2009/089260; WO 2013/142796; and WO 2013/169971. Variants of these antibodies having enhanced ADCC (i.e. ADCC greater than ADCC of ipilimumab) may find use in the methods of the present invention.

The present invention is further illustrated by the following examples, which should not be construed as limiting. The contents of all figures and all references, patents and published patent applications cited throughout this application are expressly incorporated herein by reference.

### Example 1

### Anti-CTLA-4 Vaccine Adjuvant Experiments in Cynomolgus Macaque

Experiments were performed in Mafa-A1*063+ Mauritian cynomolgus macaques (Macaca fascicularis; MCM) to track the effects of anti-CTLA-4 antibody variants differing in ADCC activity on the immune modulation of vaccine-induced antigen-specific T-cell responses over time. Three different anti-CTLA-4 monoclonal antibodies were studied: ipilimumab (ipi), nonfucosylated ipilimumab (ipi-NF), and ipilimumab having an N297A mutation (ipi-N297A), which completely blocks N-linked glycosylation. The nonfucosylated ipilimumab exhibits enhanced ADCC, whereas the N297A ipilimumab exhibits reduced/eliminated ADCC, compared with ipilimumab.

Viral vaccine immunogens were constructed by introducing the genes for simian immunodeficiency virus (SIV) Gag and Nef proteins into adenovirus serotype 5 (Ad5) vectors. The Nef gene sequence was modified to remove the second and third amino acid residues (Gly-Gly) to remove a myristolation site. Gag-Ad5 and Nef-Ad5 viruses were administered (3×10⁹ viral particles/MCM) intramuscularly in opposite hind legs to help avoid immunodominance. 3×10⁹ viral particles/MCM represents sub-optimal dosing, which was chosen to maximize the chances of observing enhanced adjuvant activity. The animals were then immediately treated (intravenously) with i) saline, ii) ipi (1 mg/kg or 10 mg/kg), iii) ipi-NF (1 mg/kg and 10 mg/kg), or iv) ipi-N297A (10 mg/kg). Blood samples were taken at days 4, 8, 15, 22, 36, and 43. Experiments were repeated twice more, except that there were 6 animals per group rather than 4, and the 1 mg/kg dose was not used, in the later experiments. The later experiments included a blood sample at day 3, and used day 36 rather than day 35. In addition, the first experiment, but not the second and third, used an allotypic variant of ipilimumab for the ipi-NF antibody comprising D357E and L359M changes relative to the heavy chain of ipilimumab (SEQ ID NO: 11).

### Whole Blood FACS to Detect Antigen-Specific T Cells

T-cell responses specific to several SIV-specific epitopes (Nef RM9, Nef LT9, Gag GW9) within the Ad5 vaccine were determined using peptide-loaded MHC class I tetramers at days 8 (day 8 only in Replicate A), 15, 22, 36 and 43, using a whole blood fluorescence-activated cell sorting (FACS) assay. Nef RM9=RPKVPLRTM=SEQ ID NO: 25; Nef LT9=LNMADKKET=SEQ ID NO: 26; Gag GW9=GPRKPIKCW=SEQ ID NO: 27. Peptide (RM9/GW9/LT9)-loaded MHC-I tetramers were used to detect antigen-specific T cells by whole blood FACS. Results are provided at FIGS. 1A-1C, 2A-2C, and 3A-3C, which provide results for SIV epitopes Nef RM9, Gag GW9 and Nef LT9, respectively. In each replicate, and for each epitope, ipi-NF at 10 mg/kg generates the highest percentages of antigen-specific CD8⁺ T cells. CD8⁺ T cells specific for Nef LT9 peak and begin to fade more rapidly than those specific for the epitopes Nef RM9 and Gag GW9, which peak around day 22 to day 36.

### ELISPOT Assay to Detect Antigen-Induced IFN-γ Production in PBMC

Enzyme-linked immunospot (ELISPOT) assays were performed on Ficoll-isolated peripheral blood mononuclear cells (PBMC) isolated from 22 day and 43 day blood samples to determine the level of IFN-γ expressed in response to antigen stimulation. PBMC were stimulated for 18 hours with 10 μM minimal optimal SIV epitope peptides. Spot-forming cell (SPC) values were measured and a background value was subtracted.

Results are provided at FIGS. 4A-4C, 5A-5C and 6A-6B, which provide results for stimulation with SIV epitopes Nef RM9, Gag GW9 and Nef LT9, respectively. The ELISPOT assays confirmed that 10 mg/kg ipi-NF treatment elicited the highest IFN-γ production in all replicates and for all SIV epitopes.

### Bulk T Cell Proliferation

CD8⁺ T cells and CD4⁺ T cells were also measured in flow cytometry on Ki-67 expression to measure cellular proliferation. Results are provided at FIGS. 7A-7C and 8A-8C. 10 mg/kg ipi-NF treatment enhanced proliferation in all replicates.

### ELISPOT Assay to Detect Ad5-Induced IFN-γ Production in PBMC

ELISPOT assays similar to those described above, were used to measure Ad5-induced IFN-γ production. Heat-inactivated Ad5 virions (5×10⁸ virus particles) were incubated for 18 hours with day 22 PBMC or day 43 PBMC. Spot-forming cell (SPC) values were measured and a background value was subtracted. Results are provided at FIGS. 9A-9C. Similar to the results obtained with SIV antigens shown in FIGS. 4A-4C, 5A-5C and 6A-6B, 10 mg/kg anti-CTLA-4-NF treatment consistently elicited the highest IFN-γ production at both 22 days and 43 days in all replicates.

In all assays tested, anti-CTLA-4-NF enhanced immune response, against three distinct SIV antigens and against Ad5 antigens generally, in this cyno vaccine model. The nonfucosylated antibody consistently generated immune responses that were both higher in magnitude and more robust than those observed with ipilimumab.

### T_{reg} Depletion

The frequency of circulating T_{regs} in the blood of cynomolgus macaques was determined by whole blood FACS assay. Samples from the animals of Replicate B were sorted to determine the frequency of T_{regs} over time as a function of which antibodies had been administered. Results are provided at FIG. 11. The anti-CTLA-4 mAb with enhanced ADCC, ipilimumab-NF, does not exhibit enhanced T_{regs} depletion compared with ipilimumab, and in fact is not significantly different from vehicle control.

### Example 2

### Anti-CTLA-4 Antibody with Enhanced ADCC Measured by Promotion of NK-Mediated Cell Lysis Using Primary Human Cells

Nonfucosylated ipilimumab was tested for its ability to promote NK cell-mediated lysis of T_{regs} from a human donor as follows. Briefly, T_{regs} for use as target cells were separated by negative selection using magnetic beads and activated for 72 hours. NK cells for use as effectors from a human donor were separated by negative selection using magnetic beads and activated with IL-2 for 24 hrs. Calcein-labeled activated T_{regs} (Donor Leukopak AC8196) were coated with various concentrations of ipilimumab, ipilimumab-NF or an IgG1 control for 30 minutes, and then incubated with NK effector cells at a ratio of 10:1 for 2 hours. Calcein release was measured by reading the fluorescence intensity of the media using an Envision plate reader (Perkin Elmer), and the percentage of antibody-dependent cell lysis was calculated based on mean fluorescence intensity (MFI) with the following formula: [(test MFI—mean background)/(mean maximum—mean background)]×100. Results are presented at FIG. 10. Nonfucosylated ipilimumab induced lysis of activated T_{regs} at an EC₅₀ (0.0065 μg/ml) significantly lower than ipilimumab (1.5 μg/ml).

### Example 3

### Assay to Determine Percentage Nonfucosylated in a Sample of Anti-CTLA-4 Antibodies

Nonfucosylated anti-CTLA-4 mAb preparations are analyzed to determine the percentage of nonfucosylated heavy chains essentially as follows.

Antibodies are first denatured using urea and then reduced using DTT (dithiothreitol). Samples are then digested overnight at 37° C. with PNGase F to remove N-linked glycans. Released glycans are collected, filtered, dried, and derivatization with 2-aminobenzoic acid (2-AA) or 2-aminobenzamide (2-AB). The resulting labeled glycans are then resolved on a HILIC column and the eluted fractions are quantified by fluorescence and dried. The fractions are then treated with exoglycosidases, such as α(1-2,3,4,6) fucosidase (BKF), which releases core α(1,6)-linked fucose residues. Untreated samples and BKF-treated samples are then analyzed by liquid chromatography. Glycans comprising α(1,6)-linked fucose residues exhibit altered elution after BKF treatment, whereas nonfucosylated glycans are unchanged. The oligosaccharide composition is also confirmed by mass spectrometry. See, e.g., Zhu et al. (2014) MAbs 6:1474.

Percent nonfucosylation is calculated as one hundred times the molar ratio of (glycans lacking a fucose α1,6-linked to the first GlcNac residue at the N-linked glycan at N297 (EU numbering) of the antibody heavy chain) to (the total of all glycans at that location (glycans lacking fucose and those having α1,6-linked fucose)).

TABLE 3
Summary of the Sequence Listing

| SEQ ID NO. | Description |
|---|---|
| 1 | human CTLA-4 (NP 005205.2) |
| 2 | human CD28 (NP 006130.1) |
| 3 | ipilimumab CDRH1 |
| 4 | ipilimumab CDRH2 |
| 5 | ipilimumab CDRH3 |
| 6 | ipilimumab CDRL1 |
| 7 | ipilimumab CDRL2 |
| 8 | ipilimumab CDRL3 |
| 9 | ipilimumab heavy chain variable domain |
| 10 | ipilimumab light chain variable domain |
| 11 | ipilimumab heavy chain |
| 12 | ipilimumab heavy chain lacking C-terminal K |
| 13 | ipilimumab light chain |
| 14 | tremelimumab CDRH1 |
| 15 | tremelimumab CDRH2 |
| 16 | tremelimumab CDRH3 |
| 17 | tremelimumab CDRL1 |
| 18 | tremelimumab CDRL2 |
| 19 | tremelimumab CDRL3 |
| 20 | tremelimumab heavy chain variable domain |
| 21 | tremelimumab light chain variable domain |
| 22 | tremelimumab heavy chain |
| 23 | tremelimumab heavy chain lacking C-terminal K |
| 24 | tremelimumab light chain |
| 25 | Nef RM9 = RPKVPLRTM |
| 26 | Nef LT9 = LNMADKKET |
| 27 | Gag GW9 = GPRKPIKCW |
| 28 | IgG1za constant domain |
| 29 | IgG1za constant domain lacking C-terminal K |

With regard to antibody sequences, the Sequence Listing provides the sequences of the mature variable regions and heavy and light chains, i.e. the sequences do not include signal peptides.

### EQUIVALENTS

Those skilled in the art will recognize, or be able to ascertain using no more than routine experimentation, many equivalents of the specific embodiments disclosed herein. Such equivalents are intended to be encompassed by the following claims.

What is claimed is:

1. A method of enhancing immune response to a vaccine in a human subject treated with the vaccine comprising administering to the subject an anti-human CTLA-4 antibody having at least twice the ADCC activity of ipilimumab.

2. The antibody of claim 1 wherein the antibody comprises:
a. a CDRH1 consisting of the sequence of SEQ ID NO: 3;
b. a CDRH2 consisting of the sequence of SEQ ID NO: 4;
c. a CDRH3 consisting of the sequence of SEQ ID NO: 5;
d. a CDRL1 consisting of the sequence of SEQ ID NO: 6;
e. a CDRL2 consisting of the sequence of SEQ ID NO: 7; and
f. a CDRL3 consisting of the sequence of SEQ ID NO: 8.

3. The antibody of claim 2 wherein the antibody comprises:
a. a heavy chain variable domain consisting of the sequence of SEQ ID NO: 9; and
b. a light chain variable domain consisting of the sequence of SEQ ID NO: 10.

4. The antibody of claim 3 wherein the antibody comprises:
a. a heavy chain consisting of the sequence of SEQ ID NO: 12; and
b. a light chain consisting of the sequence of SEQ ID NO: 13.

5. The antibody of claim 4 wherein the antibody comprises:
a. a heavy chain comprising the sequence of SEQ ID NO: 11; and
b. a light chain comprising the sequence of SEQ ID NO: 13.

6. The method of claim 1 wherein the anti-human CTLA-4 antibody having at least twice the ADCC activity of ipilimumab exhibits an EC50 for cell lysis that is at least two-fold lower than the EC50 for cell lysis for ipilimumab in the NK92 cell mediated lysis assay detailed in Example 2.

7. The method of claim 6 wherein the anti-human CTLA-4 antibody having at least twice the ADCC activity of ipilimumab exhibits an EC50 for cell lysis that is at least ten-fold lower than the EC50 for cell lysis for ipilimumab in the NK92 cell mediated lysis assay detailed in Example 2.

8. The antibody of claim 1 wherein the antibody comprises:
a. a CDRH1 consisting of the sequence of SEQ ID NO: 14;
b. a CDRH2 consisting of the sequence of SEQ ID NO: 15;
c. a CDRH3 consisting of the sequence of SEQ ID NO: 16;
d. a CDRL1 consisting of the sequence of SEQ ID NO: 17;
e. a CDRL2 consisting of the sequence of SEQ ID NO: 18; and
f. a CDRL3 consisting of the sequence of SEQ ID NO: 19.

9. The antibody of claim 8 wherein the antibody comprises:
a. a heavy chain variable domain consisting of the sequence of SEQ ID NO: 20; and
b. a light chain variable domain consisting of the sequence of SEQ ID NO: 21.

10. The antibody of claim 9 wherein the antibody comprises:
a. a heavy chain consisting of the sequence of SEQ ID NO: 23; and
b. a light chain consisting of the sequence of SEQ ID NO: 24.

11. The antibody of claim 9 wherein the antibody comprises:
a. a heavy chain comprising the sequence of SEQ ID NO: 22; and
b. a light chain comprising the sequence of SEQ ID NO: 24.

12. The method of claim 1 wherein the anti-human CTLA-4 antibody having at least twice the ADCC activity of ipilimumab exhibits an EC50 for cell lysis that is at least two-fold lower than the EC50 for cell lysis for ipilimumab in the NK92 cell mediated lysis assay detailed in Example 2.

13. The method of claim 12 wherein the anti-human CTLA-4 antibody having at least twice the ADCC activity of ipilimumab exhibits an EC50 for cell lysis that is at least ten-fold lower than the EC50 for cell lysis for ipilimumab in the NK92 cell mediated lysis assay detailed in Example 2.

14. The method of claim 1 wherein the anti-human CTLA-4 antibody having at least twice the ADCC activity of ipilimumab has reduced fucosylation.

15. The method of claim 14 wherein the anti-human CTLA-4 antibody having at least twice the ADCC activity of ipilimumab is hypofucosylated or nonfucosylated.

16. The method of claim 15 wherein the anti-human CTLA-4 antibody having at least twice the ADCC activity of ipilimumab is nonfucosylated.

17. The method of claim 1 wherein the anti-human CTLA-4 antibody having at least twice the ADCC activity of ipilimumab comprises an IgG1 heavy chain constant region comprising a mutation, or cluster of mutations, selected from the group consisting of: i) G236A; ii) S239D; iii) F243L; iv) E333A; v) G236A/I332E; vi) S239D/I332E; vii) S267E/H268F; viii) S267E/S324T; ix) H268F/S324T; x) G236A/S239D/I332E; xi) S239D/A330L/I332E; xii) S267E/H268F/S324T; and xiii) G236A/S239D/A330L/I332E.

18. The method of claim 17 wherein the anti-human CTLA-4 antibody having at least twice the ADCC activity of ipilimumab has reduced fucosylation.

19. The method of claim 18 wherein the anti-human CTLA-4 antibody having at least twice the ADCC activity of ipilimumab is hypofucosylated or nonfucosylated.

20. The method of claim 19 wherein the anti-human CTLA-4 antibody having at least twice the ADCC activity of ipilimumab is nonfucosylated.
